(19) United States
(12) Patent Application Publication
(10) Pub. No.: US 2022/0288299 A1
(43) Pub. Date: Sep. 15, 2022

(54) PEN NEEDLE MAGAZINE

(71) Applicant: Becton, Dickinson and Company, Franklin Lakes, NJ (US)

(72) Inventors: Amit Limaye, Wayne, NJ (US); Dipumon Ayyanchira Mani, Kerala (IN); Praveesh Karattu Meethal, Kerala (IN); Sajayesh Vijayachandran, Kerala (IN)

(73) Assignee: Becton, Dickinson and Company, Franklin Lakes, NJ (US)

(21) Appl. No.: 17/615,500

(22) PCT Filed: Apr. 26, 2019
(86) PCT No.: PCT/US2019/029321

§ 371 Date: Nov. 30, 2021

(51) Int. Cl. A61M 5/00; A61M 5/32; A61M 5/50; A61M 5/34

(52) U.S. Cl. CPC A61M 5/002; A61M 5/3245; A61M 5/5086; A61M 5/347; A61M 2005/004; A61M 2005/3254; A61M 2005/3208

(57) ABSTRACT

A pen needle magazine (10) comprising a plurality of compartments (14) each carrying a pen needle (40) with an inner thread (44) and an outer thread (46), and an inner shield (80) including an inner thread (82), the inner shield (80) being disposed in each of the plurality of compartments (14), wherein the inner thread (82) of the inner shield (80) is configured to engage the outer thread (46) of the pen needle (40), and the inner thread (44) and the outer thread (46) of the pen needle (40) are threaded in different directions.

### FIELD OF THE INVENTION

Various exemplary embodiments of the invention relate to pen needle storage and disposal for medication delivery pens.

### BACKGROUND OF THE INVENTION

Medication pens are typically used to inject medication into a patient. A person who must periodically self-inject doses of medication will typically carry a medication pen, several single-use pen needles, and several cleaning swabs. A medication pen is designed for safety and sterility. However, inefficiencies and inconveniences can arise.

### SUMMARY OF THE INVENTION

It is an aspect of the present invention to provide a pen needle magazine that stores a plurality of needles each configured to engage a medication pen for medication delivery. Such a magazine provides advantages in easy organization, allows for needle disposal after use and storage before use in the same magazine and improves usage with an optimized workflow to reduce setup time and space. Needle disposal after use and storage before use allows for synchronized pen needle attachment and removal, as well as synchronized pen needle detachment and disposal. Finally, the controlled movement of the pen needle attachment and removal from the magazine reduces user needle sticking and reduces accidental bending of a needle in the pen needle.

The pen needle magazine also reduces the likelihood of pen needle reuse in several ways. First, the pen needle magazine has a seal tab that acts as a visual indicator to indicate to a user whether the pen needle is used or new. Second, the seal tab is disposed in a tamper resistant member and cooperates with a locking mechanism to open the compartment. Finally, after the seal tab is removed, the tamper resistant member prevents access to the locking mechanism between the compartment door and the respective compartment.

The foregoing and/or other aspects of the present invention can be achieved by providing a pen needle magazine comprising a plurality of compartments each having a stepped locking element, the plurality of compartments each carrying a pen needle, a seal configured to be disposed on a top surface of each of the plurality of compartments to enclose each of the pen needles in a corresponding compartment, and a plurality of compartment doors each having a snap locking member that is configured to engage the stepped locking element, the plurality of compartment doors each configured to cover a corresponding compartment, wherein in a closed, unlocked position of one of the plurality of compartments, the seal is disposed between the top surface of one of the plurality of compartments and a corresponding compartment door, and the seal is disposed between the stepped locking element and the snap locking member.

The foregoing and/or other aspects of the present invention can further be achieved by providing a pen needle magazine comprising a plurality of compartments each carrying a pen needle with an inner thread and an outer thread, and an inner shield including an inner thread, the inner shield being disposed in each of the plurality of compartments, wherein the inner thread of the inner shield is configured to engage the outer thread of the pen needle, and the inner thread and the outer thread of the pen needle are threaded in different directions.

The foregoing and/or other aspects of the present invention can also be achieved by providing a pen needle magazine comprising a plurality of compartments each including an inner thread, each compartment carrying a pen needle with an inner thread and an outer thread, wherein the inner thread is configured to engage the outer thread of the pen needle, and the inner thread and the outer thread of the pen needle are threaded in different directions.

The foregoing and/or other aspects of the present invention can additionally be achieved by providing a method of using a plurality of pen needles stored in a housing, the method comprising connecting a unused pen needle to a medication delivery pen for medication delivery, removing the unused pen needle from a cavity of the housing simultaneously with the connecting step, administering medication via the pen needle connected to the medication delivery pen, disposing of the used pen needle after medication delivery into the cavity of the housing and simultaneously disconnecting the used pen needle from the medication delivery pen, and sending the housing to a manufacturer or a waste management entity after use of all of the plurality of pen needles in the housing.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 shows a perspective view of an exemplary pen needle magazine;

FIG. 2 shows a cross-sectional view of a pen assembly including a medication delivery pen and the pen needle magazine of FIG. 1;

FIG. 3 shows a cross-sectional view of a compartment in the pen needle magazine of FIG. 1 ready for use in a closed, unlocked position;

FIG. 4 shows a cross-sectional view of the compartment of FIG. 3 in an open position;

FIG. 5 shows a cross-sectional view of the medication delivery pen in the process of being engaged to the pen needle in the compartment and the pen needle in the process of being disengaged from the compartment of FIG. 4;

FIG. 6 shows a cross-sectional view of the medication delivery pen removing the pen needle from the compartment of FIG. 5;

FIG. 7 shows a cross-sectional view of the used pen needle ready for disposal in the compartment of FIG. 6;

FIG. 8 shows a cross-sectional view of the medication delivery pen in the process of being disengaged from the pen needle and the pen needle in the process of being engaged to the compartment of FIG. 7;

FIG. 9 shows a cross-sectional view of the medication delivery pen separated from the pen needle and the pen needle being disposed in the compartment of FIG. 8;

FIG. 10 shows a cross-sectional view of the compartment of FIG. 9 in a closed, locked position;

FIG. 11 shows a cross-sectional view of an alternate embodiment of a locking mechanism between the compartment of FIG. 3 and the compartment door in a closed, unlocked position; and

FIG. 12 shows a cross-sectional view of the locking mechanism of FIG. 11 between the compartment and the compartment door in a closed, locked position.

### DETAILED DESCRIPTION OF EXEMPLARY EMBODIMENTS

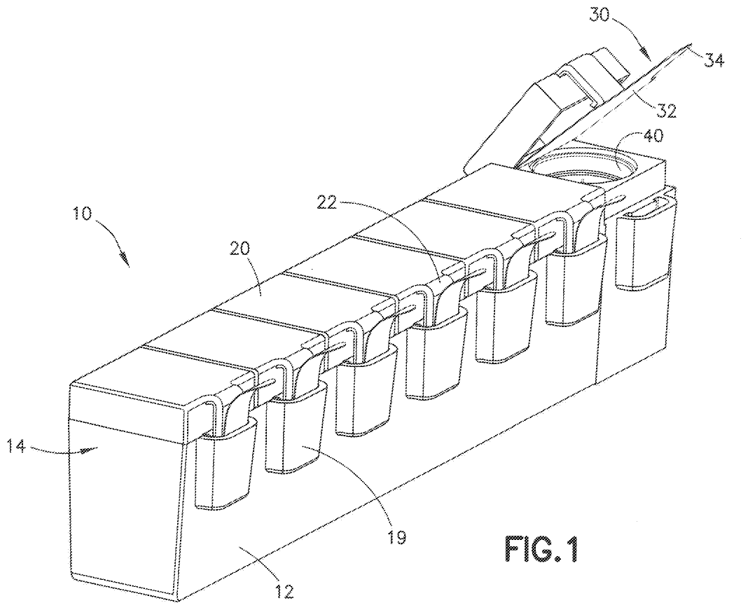

According to one embodiment, FIG. 1 illustrates a pen needle magazine 10 configured to carry a plurality of pen needles 40. The pen needle magazine 10 includes a magazine housing 12, a plurality of compartments 14 and a plurality of compartment doors 20. The magazine housing 12 is a substantially rectangular shaped linear array of adjacent compartments 14. Each of the plurality of compartments 14 is substantially square shaped and includes a cavity to carry a pen needle 40. Each of the plurality of compartment doors 20 is hinged on one side (rear surface) of each of the compartments 14 and is configured to open and close to cover and provide access to a corresponding compartment 14 of the plurality of compartments 14. Such a configuration advantageously provides an easy organization of pen needles 40.

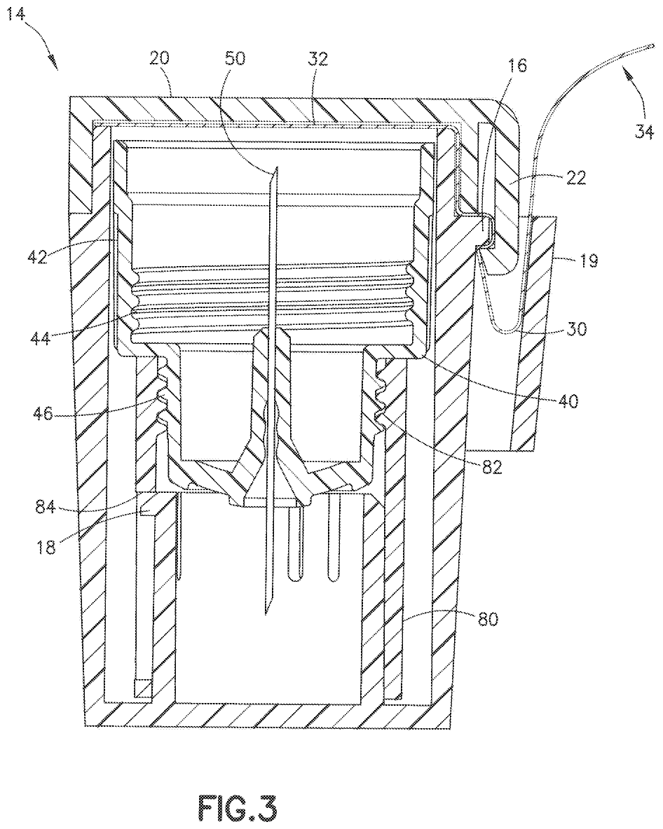

The plurality of compartments 14 each further includes a stepped locking element 16, a flanged locking element 18 and a tamper resistant member 19. The stepped locking element 16, as illustrated in FIG. 3, is a protruded surface extending beyond an external side surface (front surface) of each compartment 14. The stepped locking element 16 is also disposed near the proximal end of the compartment 14 and is configured to engage and lock to the compartment door 20. Further, the stepped locking element 16 is disposed on an exterior side surface of each compartment 14 opposite to a side surface with the hinge. Further operational details of the stepped locking element 16 are described below.

The flanged locking element 18 is a circular element disposed on an inner bottom surface at a distal end of each of the plurality of compartments 14. The flanged locking element 18 extends upwardly from the bottom surface and is configured to interact with an inner shield 80. A plurality of flanges are partially cut out of the flanged locking element 18 to provide increased flexibility and elastic movement. The plurality of flanges extend upwardly and radially outward at a proximal end and along a circumferential surface. These flanges engage the inner shield 80 to lock the inner shield 80 to the compartment 14. The increased flexibility and elastic movement allow for the engagement of the inner shield 80 to the compartment 14.

The tamper resistant member 19 is a hollow cavity that protrudes from the front surface of each of the compartments 14. Specifically, the tamper resistant member 19 includes a protruding member with a hollow opening disposed at an external side surface of each of the plurality of compartments 14. The hollow opening has a space for the stepped locking element 16 of the compartment 14 to engage a snap locking member 22 of the compartment door 20.

Additionally, a seal 30 is configured to be disposed between the stepped locking element 16 and the snap locking member 22 in a closed, unlocked position of the compartment 14. The tamper resistant member 19 advantageously prevents the user from manipulating the stepped locking element 16 and the snap locking member 22, particularly in the closed, locked position. The tamper resistant member 19 also partially shields the seal 30, particularly at a bent portion of the seal 30, when the compartment 14 is in the closed, unlocked position. Details of the snap locking member 22 and the seal 30 are further described below.

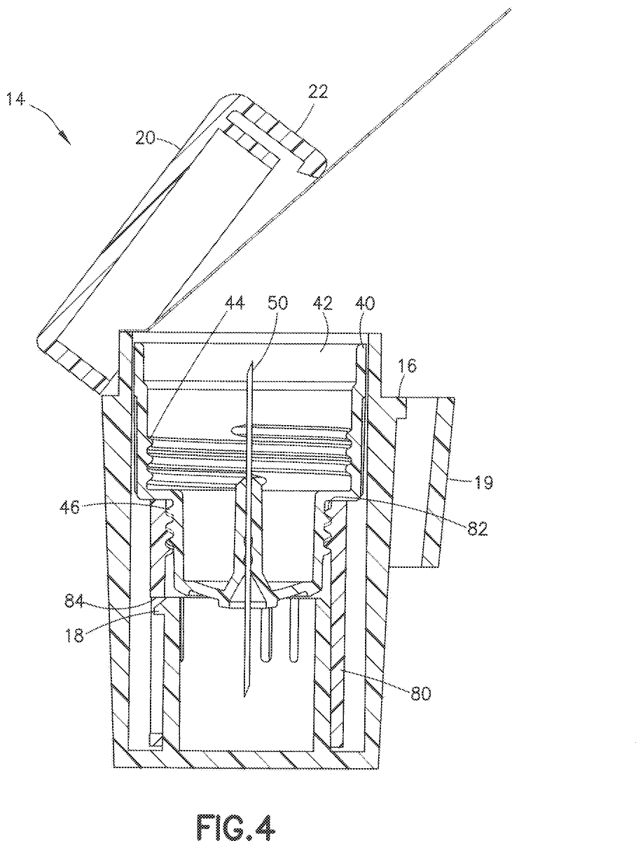

FIGS. 3 and 4 illustrate the snap locking member 22 of each of the compartment doors 20. The snap locking member 22 is a flanged member disposed at the side surface (adjacent to the front surface of the compartment 14) opposite to the hinged surface (adjacent to the rear surface of the compartment 14). The snap locking member 22 is configured to engage and lock to the stepped locking element 16 of the compartment 14 (locking mechanism). Specifically, a flanged surface of the snap locking member 22 is configured to engage a bottom surface of the stepped locking element 16. However, the seal 30 can be disposed between the snap locking member 22 and the stepped locking element 16 to keep the compartment door 20 in the closed, yet unlocked position (closed, unlocked position).

Before the pen needles 40 are accessed from the plurality of compartments 14, each of the plurality of compartments 14 is enclosed by a seal 30. FIG. 1 illustrates a plurality of seals 30 each enclosing one of the plurality of compartments 14. The compartment 14 on the far right of FIG. 1 is opened with the seal 30 removed from a top surface of the compartment 14.

The seals 30 are composed of any combination of a plastic, polymer, foil or metallic material, or a composite polymeric metallic multi-laminate. The material of the seals 30 provides a hermetic or air tight seal for the cavity in the compartment 14. In addition, the material advantageously allows the seal 30 to be flexible and adjustable in a variety of shapes without plastic deformation or fracture.

The seal 30 at the far right in FIG. 1 best illustrates the components of the seal 30. Specifically, the seal 30 includes a sealing portion 32 and a tab portion 34. The sealing portion 32 is disposed directly above the top surface of the respective compartment 14 to enclose the pen needle 40 in the compartment 14. When the compartment door 20 is secured to the magazine housing 12, the compartment door 20 covers the sealing portion 32 to advantageously prevent accidental puncturing. Thus, the sealing portion 32 is sandwiched between the top surface of the compartment 14 and the compartment door 20 in the closed, unlocked position of the compartment 14.

The tab portion 34 is adjacent to the corresponding compartment 14. In the closed, unlocked position, as illustrated in FIG. 3, the tab portion 34 is disposed between the compartment door 20 and the compartment 14. Specifically, the tab portion 34 is disposed along the front surface of the compartment 14, and between the stepped locking element 16 of the compartment 14 and the snap locking member 22 of the compartment door 20. Positioning the tab portion 34 in this manner prevents engagement of the stepped locking element 16 to the snap locking member 22. The material of the tab portion 34 advantageously provides rigidity to prevent engagement of the stepped locking element 16 to the snap locking member 22.

The tab portion 34 is then bent upwardly in an angle close to 180° at the bent portion and exits the proximal end of the tamper resistant member 19. In other words, the tab portion 34 enters and exits at the proximal end of the tamper resistant member 19. Accordingly, the user is able to advantageously access and use the tab portion 34 to flex the snap locking member 22 outward and open the compartment door 20 in an opened, unlocked position. Specifically, the user holds the tab portion 34 and pulls upward to open the compartment door 20 and remove the sealing portion 32 from the top surface of the compartment 14.

The tab portion 34 also acts as a visual indicator for the user outside of the closed compartment door 20 to indicate that the pen needle 40 is enclosed in the compartment 14 by the seal 30. Accordingly, such a configuration advantageously indicates that the pen needle 40 is unused and ready for use.

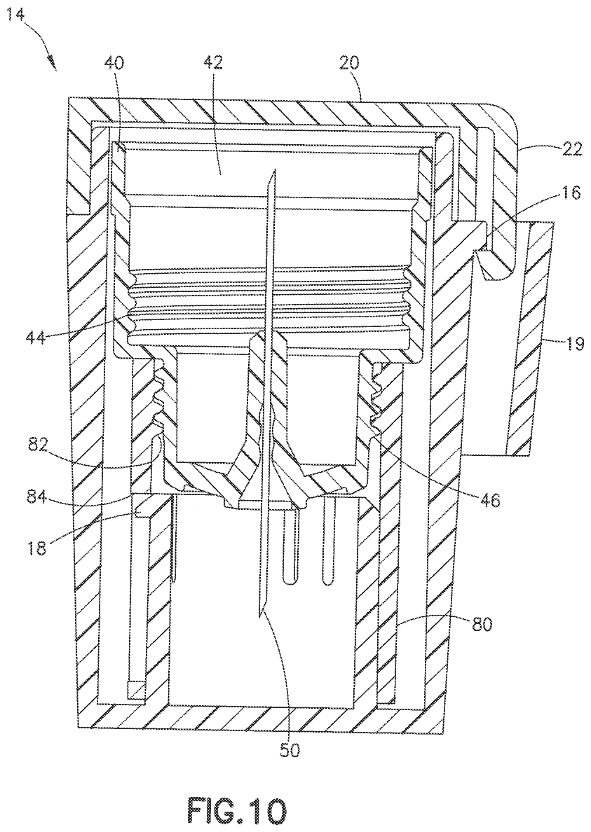

FIG. 10 illustrates one of the compartments 14 in a closed, locked position where the used pen needle 40 is returned to the compartment 14 and the compartment door 20 is closed. In this configuration, the seal 30 is no longer present and so the stepped locking element 16 of the compartment 14 engages the snap locking member 22 of the compartment door 20 to lock the used pen needle 40 in the compartment 14.

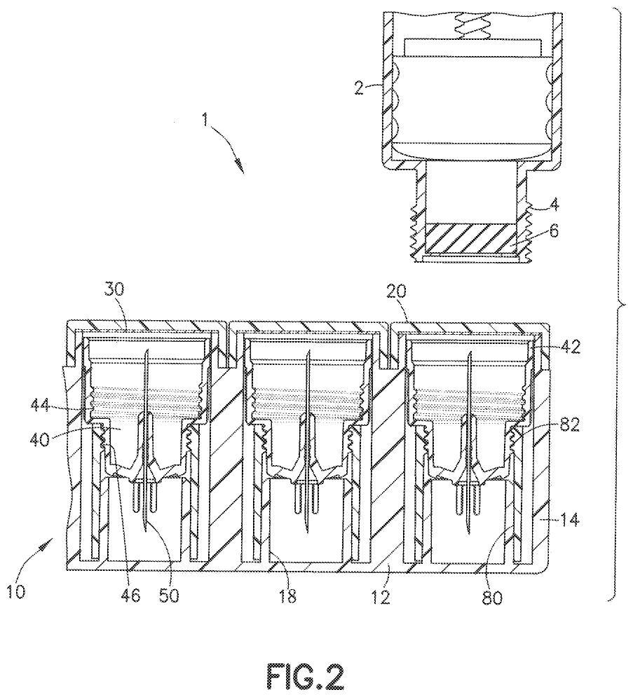

FIG. 2 illustrates a pen assembly 1 including the pen needle magazine 10 and a medication delivery pen 2. The medication delivery pen 2 includes an external thread 4 and a septum 6. The external thread 4 is configured to thread into and engage an inner thread 44 of the pen needle 40. The septum 6 is a seal that holds medicament inside a cartridge of the medication delivery pen 2. When the septum 6 is pierced by a non-patient end of a needle 50 of the pen needle 40, fluid communication is established between the medication delivery pen 2 and a patient-end of the needle 50 of the pen needle 40.

FIGS. 2 and 3 illustrates the pen needle 40. The pen needle 40 includes a hub 42, the inner thread 44, an outer thread 46, and the needle 50. The hub 42 is the housing or base of the pen needle 40. As described above, the inner thread 44 is configured to attach to the outer thread 4 of the medication delivery pen 2. Upon full engagement of the inner thread 44 to the outer thread 4, the non-patient end of the needle 50 pierces the septum 6 of the medication delivery pen 2 to establish fluid communication.

The outer thread 46 of the pen needle 40 is configured to engage an inner thread 82 of the inner shield 80. Specifically, the outer thread 46 is advantageously threaded in an opposite direction that the inner thread 44. In this manner, the pen needle 40 provides simultaneously engagement and disengagement of the medication delivery pen 2 and the inner shield 80 during pen needle 40 attachment and removal, as well as during pen needle 40 detachment and disposal.

In other words, the inner thread 44 and the outer thread 46 of the pen needle 40 are advantageously threaded in different directions. Such a configuration advantageously synchronizes pen needle 40 attachment and removal with pen needle 40 detachment and disposal. Specifically, when the thread 4 of the medication delivery pen 2 engages the inner thread 44 of the pen needle 40, the outer thread 46 of the pen needle 40 simultaneously disengages from the inner thread 82 of the inner shield 80. This simultaneous action occurs during the same rotational movement to remove the pen needle 40 from the compartment 14 for use. Also, when the thread 4 of the medication delivery pen 2 disengages from the inner thread 44 of the pen needle 40, the outer thread 46 of the pen needle 40 simultaneously engages the inner thread 82 of the inner shield 80. This simultaneous action occurs during the same rotational movement to dispose the used pen needle 40 into the compartment 14 for disposal.

In one embodiment, the inner thread 44 of the pen needle 40 is threaded in a clockwise direction and the outer thread 46 of the pen needle 40 is threaded in a counterclockwise direction from a top view looking downward on the pen assembly 1 of FIG. 2. In another embodiment, the inner thread 44 of the pen needle 40 is threaded in a counterclockwise direction and the outer thread 46 of the pen needle 40 is threaded in a clockwise direction from a top view looking downward on the pen assembly 1 of FIG. 2.

Finally, the pen needle 40 includes the needle 50 as conventionally understood by one of ordinary skill in the art. When the pen needle 40 is attached to the medication delivery pen 4, the proximal end of the needle 50 (non-patient end) pierces the septum 6 of the medication delivery pen 4 to establish fluid communication. Accordingly, during use, the distal end (patient end) of the needle 50 provides fluid communication between the medication delivery pen 2 and the patient to administer the medicament.

FIGS. 2 and 3 illustrate the preferred embodiment of the inner shield 80 having the inner thread 82 and lock openings 84. The inner shield 80 is a hollow cylinder disposed at a distal end of each of the compartments 14. The inner thread 82 is disposed at a proximal end on an inner surface of the inner shield 80 and is configured to engage the outer thread 46 of the pen needle 40.

The inner shield 80 surrounds the circular feature of the flanged locking element 18 in the compartment 14. The lock openings 84 comprise vertical slots that engage the flanges of the flanged locking element 18 to rotationally and axially lock the inner shield 80 to the compartment 14. Specifically, the flanges of the flanged locking element 18 extend outwardly to extend into the lock openings 84 of the inner shield 80 and lock. Upon engagement, the inner shield 80 is unable to be detached from the compartment 14. In addition, the inner shield 80 is unable to rotate within the compartment 14.

In an alternate embodiment, the inner shield 80 having the inner thread 82 and the lock openings 84 are an integral portion of each of the compartments 14. In this configuration, the features of the inner shield 80 function in a similar manner as described throughout this application.

Detailed operation of the pen needle magazine 10 is described as follows. Throughout operation, one of the inner shields 80 is locked to each of the plurality of compartments 14. Specifically, the flange-locking element 18 of the compartment 14 is engaged to the lock openings 84 in the inner shield 80. This engagement prevents the inner shield 80 from moving rotationally in the compartment 14 and being removed from the compartment 14.

FIG. 3 illustrates one of the plurality of compartments 14 in the closed, unlocked position. In this position, the compartment 14 carrying an unused pen needle 40 is threaded to the inner shield 80 via the outer thread 46 of the pen needle 40 and the inner thread 82 of the shield 80. The sealing portion 32 of the seal 30 encloses the compartment 40 and the compartment door 20 is advantageously disposed over the seal 30 to further cover the compartment 14 and protect the seal 30 from inadvertent puncture.

The tab portion 34 is disposed between the stepped locking element 16 of the compartment 14 and the snap locking member 22 of the compartment door 20. The tab portion 34 prevents the compartment door 20 from being locked to the compartment 14 by preventing engagement of the snap locking member 22 to the stepped locking element 16.

The tab portion 34 is routed in a downward direction to be disposed between the snap locking member 22 to the stepped locking element 16. Subsequently, the tab portion 34 is bent upward at approximately 180° to be accessible outside the tamper resistant member 19 of the compartment 14. A portion of the tab portion 34 and the interface between the snap locking member 22 and the stepped locking element 16 are disposed in the tamper resistant member 19 to advantageously protect these features from inadvertent manipulation and use.

FIG. 4 illustrates open, unlocked position of the compartment 14. In this position, the user pulls the tab portion 34 of the seal 30 upwardly to flex the snap locking member 22 outwardly from the compartment 14 and upwardly from the tamper resistant member 19. Subsequently, the sealing portion 32 is removed from the top surface of the compartment 14. At the same time, the compartment door 20 rotates to open the compartment 14. The pen needle 40 is now exposed and ready for removal and use.

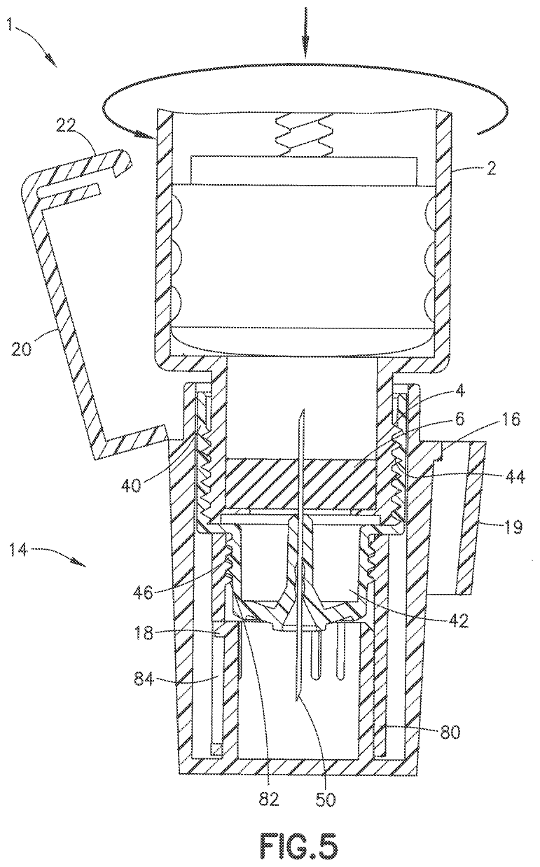

FIG. 5 illustrates the medication delivery pen 2 in the process of engaging the pen needle 40 while the pen needle 40 is in the process of disengaging the inner shield 80. Specifically, the user pushes and rotates the medication delivery pen 2 to rotate the thread 4 in a clockwise direction from a top view to thread into the inner thread 44 of the pen needle 40 and attach to the medication delivery pen 2. At the same time, the outer threads 46 of the pen needle 40 unthreads from the inner threads 82 of the inner shield 80 in a counterclockwise direction from a top view to detach from the inner shield 80 and ultimately be removed from the compartment 14. As described above, the inner shield 80 is rotationally fixed to the compartment 14 to allow for the unthreading. This synchronized attachment and removal of the pen needle 40 provides significant advantages in efficient usability and simplicity of design.

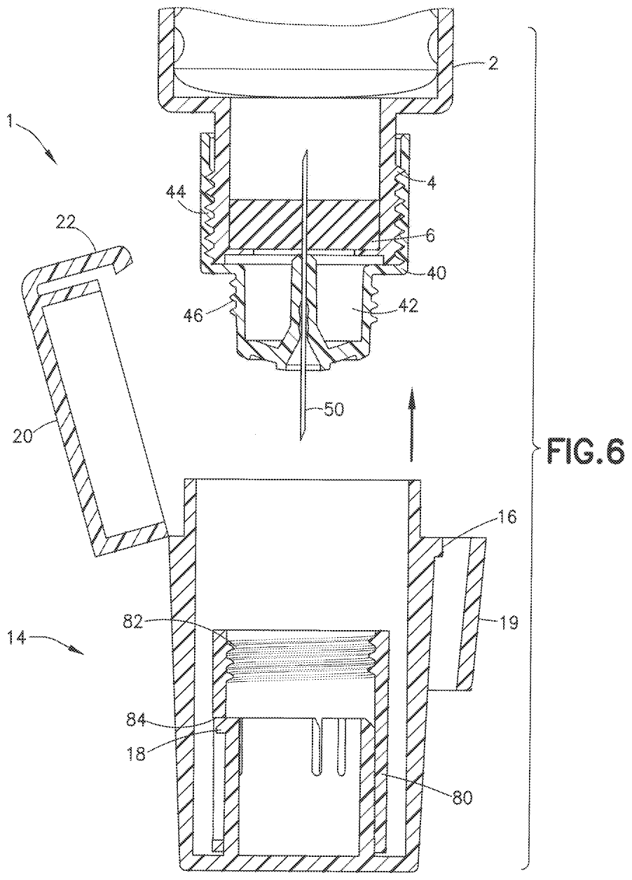

FIG. 6 illustrates the medication delivery pen 2 engaged to the pen needle 40 and removed from the compartment 14. When the pen needle 40 engages the medication delivery pen 2, the proximal end (non-patient end) of the needle 50 pierces the septum 6. Accordingly, the distal end (patient end) of the needle 50 is in fluid communication with the medication delivery pen 2 and is ready for injection and medication delivery.

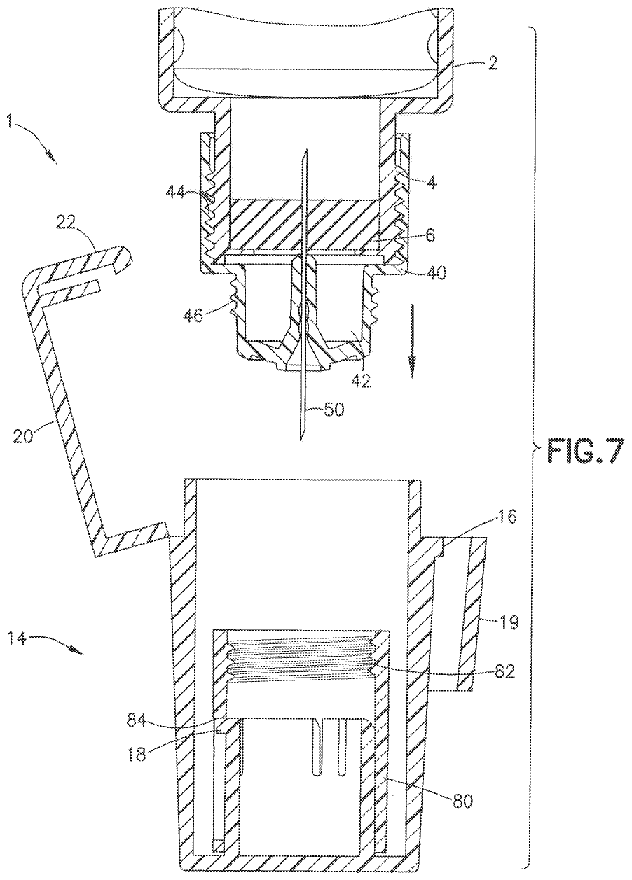

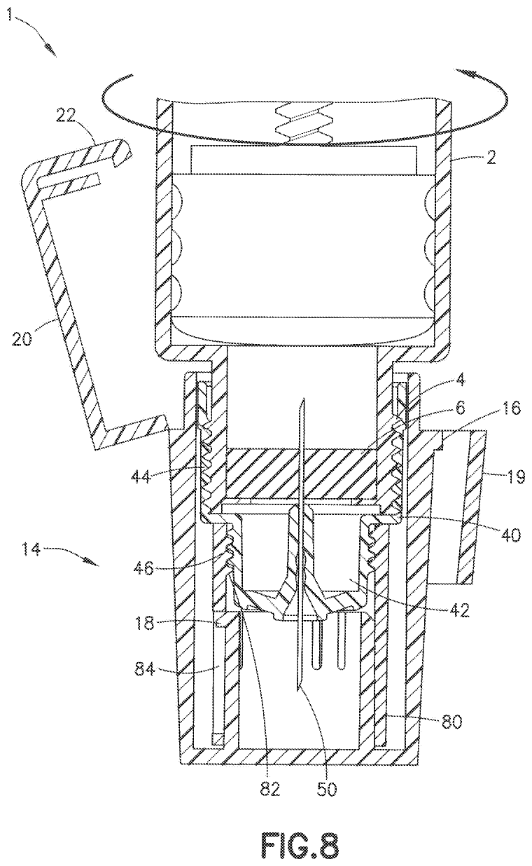

FIG. 7 illustrates the used pen needle 40 attached to the medication delivery pen 2 where the user is ready to discard the used pen needle 40 into the compartment 14. FIG. 8 illustrates the pen needle 40 in the process of disengaging from the medication delivery pen 2 while the pen needle 40 is in the process of engaging the inner shield 80. Specifically, the user pushes and rotates the pen needle 40 attached to the medication delivery pen 2 in a counterclockwise direction from a top view to unthread and detach the inner threads 44 of the pen needle 40 from the thread 4 of the medication delivery pen 2. At the same time, the thread the outer thread 46 thread into the inner thread 82 of the inner shield 80 in a clockwise direction from a top view to retain and ultimately dispose the used pen needle 40 in the compartment 14 of the magazine housing 12. As described above, the inner shield 80 is rotationally fixed to the compartment 14 to allow for the threading. This synchronized detachment and disposal of the pen needle 40 provides significant advantages in efficient usability and simplicity of design.

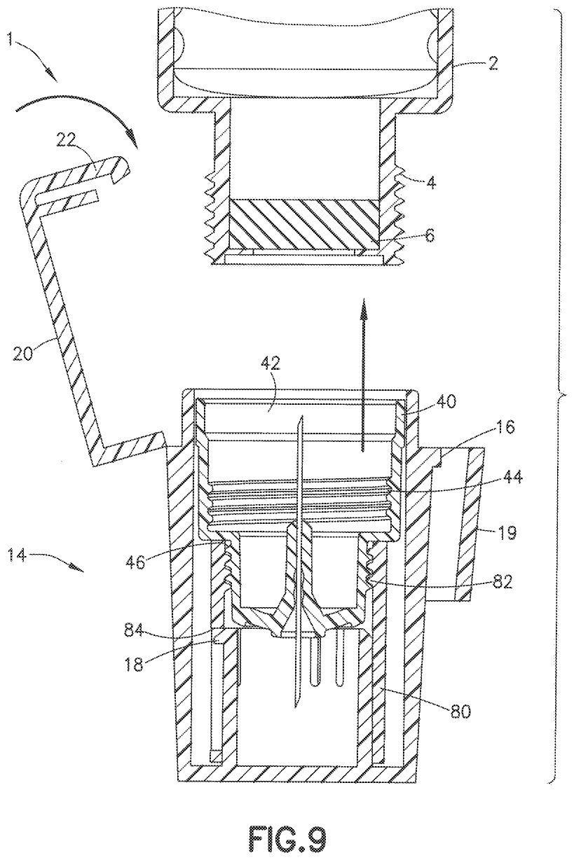

FIG. 9 illustrates the medication delivery pen 2 disengaged from the pen needle 40 where the medication delivery pen 2 is detached from the compartment 14 and the pen needle 40 is retained in the compartment 14. Fluid communication between the pen needle 40 and the medication delivery pen 2 is now terminated.

FIG. 10 illustrates the compartment door 20 closing the compartment 14 that carries the used pen needle 40. In the closed, locked position, the compartment door 20 is locked to the compartment 14. Specifically, the stepped locking element 16 of the compartment 14 engages the snap locking member 22 of the compartment door 20 to provide a secure locking arrangement. The stepped locking element 16 and the snap locking member 22 are engaged within the tamper resistant member 19 to protect this locking engagement from any tampering. The user can now use a new pen needle 40 from another compartment 14.

After all the pen needles 40 have been used, and returned to the respective compartments 14 of the pen needle magazine 10, the user can advantageously send the pen needle magazine 10, via mail, for example, to a manufacturer or a waste management entity for safe and sterile disposal. Optionally, the manufacturer can advantageously recycle and reuse various components of the pen needle magazine 10. For example, the manufacturer can sterilize and reuse some or all of the compartments 14 in the pen needle magazine 10. Specifically, the manufacturer can replace the used pen needle 40 with the unused pen needle 40 in one or more compartments 14 and seal the respective compartment 14 with the seal 30. The pen needle magazine 10 can then be shipped and ultimately sold for reuse.

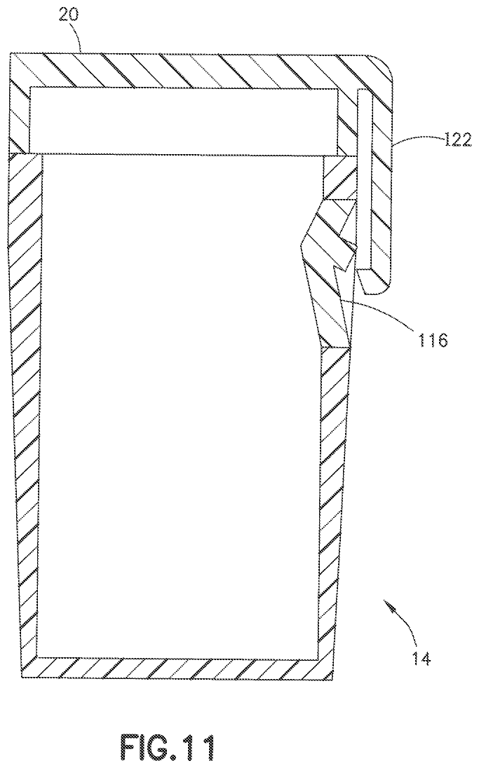

FIG. 11 illustrates an alternate embodiment of a locking mechanism comprising a stepped locking element 116 of the compartment 14 and a snap locking member 122 of the compartment door 20. The stepped locking element 116 is a frangible or flexible member that flexes or elastically deforms to disengage from the snap locking member 122. In one embodiment, the stepped locking element 116 is an insert that mates with an opening in the compartment 14. In another embodiment, stepped locking element 116 is integral to the compartment 14. This locking mechanism prevents the possibility of the user accidently closing the compartment door 20 before disposing of the used pen needle 40 in the compartment 14.

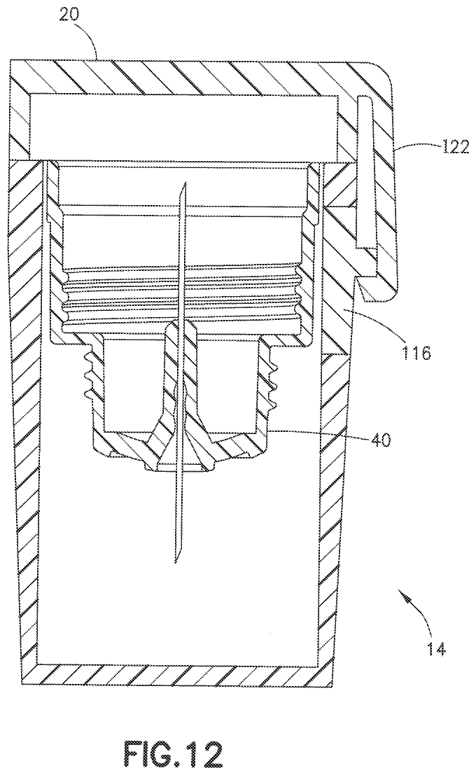

FIG. 11 illustrates the compartment door 20 in the closed, unlocked position with respect to the compartment 14. On the other hand, FIG. 12 illustrates the stepped locking element 116 being rigid to engage with the snap locking member 122. In this configuration, the compartment door 20 is in the closed, locked position with respect to the compartment 14.

The storage and use of the pen needles 40, as well as the disposal of the pen needles 40 in the same pen needle magazine 10 provides many advantages and benefits. There is no need for separate packages for storage and disposal of pen needles 40. This is because the pen needle magazine 10 advantageously synchronizes pen needle 40 attachment and removal with pen needle 40 detachment and disposal. This versatile arrangement provides better usage, optimizes workflow, minimizes setup time and optimizes space.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. In addition, any of the embodiments, features and/or elements disclosed herein may be combined with one another to form various additional combinations not specifically disclosed, as long as the embodiments, features and/or elements being combined do not contradict each other. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges around and including the given value, for example, general tolerances associated with manufacturing, assembly, and use of the described embodiments.

1. A pen needle magazine (10) comprising:
a plurality of compartments (14) each having a stepped locking element (16), the plurality of compartments (14) each carrying a pen needle (40);
a seal (30) configured to be disposed on a top surface of each of the plurality of compartments (14) to enclose each of the pen needles (40) in a corresponding compartment (14); and
a plurality of compartment doors (20) each having a snap locking member (22) that is configured to engage the stepped locking element (16), the plurality of compartment doors (20) each configured to cover a corresponding compartment (14); wherein
in a closed, unlocked position of one of the plurality of compartments (14), the seal (30) is disposed between the top surface of one of the plurality of compartments (14) and a corresponding compartment door (20), and the seal (30) is disposed between the stepped locking element (16) and the snap locking member (22).

2. The pen needle magazine (10) of claim 1, wherein in a closed, locked position of one of the plurality of compartments (14), the seal (30) is removed and the stepped locking element (16) engages the snap locking member (22).

3. The pen needle magazine (10) of claim 1, wherein in an open, unlocked position of one of the plurality of compartments (14), the seal (30) is removed from the top surface of the compartment (14) to access the pen needle (40) and simultaneously open the compartment (14).

4. A pen needle magazine (10) comprising:
a plurality of compartments (14) each carrying a pen needle (40) with an inner thread (44) and an outer thread (46); and
a plurality of inner shields (80) each including an inner thread (82), one of the inner shields (80) being disposed in each of the plurality of compartments (14); wherein
the inner thread (82) of the inner shield (80) is configured to engage the outer thread (46) of the pen needle (40); and
the inner thread (44) and the outer thread (46) of the pen needle (40) are threaded in different directions.

5. The pen needle magazine (10) of claim 4, wherein the inner thread (44) of the pen needle (40) is threaded in a clockwise direction and the outer thread (46) of the pen needle (40) is threaded in a counterclockwise direction.

6. The pen needle magazine (10) of claim 4, wherein the inner thread (44) of the pen needle (40) is threaded in a counterclockwise direction and the outer thread (46) of the pen needle (40) is threaded in a clockwise direction.

7. A pen assembly (1) comprising:
a medication delivery pen (2) have a thread (4); and
the pen needle magazine (10) of claim 4; wherein
when the thread (4) of the medication delivery pen (2) engages the inner thread (44) of the pen needle (40), the outer thread (46) of the pen needle (40) disengages from the inner thread (82) of the inner shield (80).

8. A pen assembly (1) comprising:
a medication delivery pen (2) have a thread (4); and
the pen needle magazine (10) of claim 4; wherein
when the thread (4) of the medication delivery pen (2) disengages from the inner thread (44) of the pen needle (40), the outer thread (46) of the pen needle (40) engages the inner thread (82) of the inner shield (80).

9. The pen needle magazine (10) of claim 4, wherein the inner shield (80) is disposed at a distal end of each of the plurality of compartments (14).

10. The pen needle magazine (10) of claim 4, wherein each of the plurality of compartments (14) includes a flange-locking element (18) extending from a bottom inner surface of the compartment (14).

11. The pen needle magazine (10) of claim 10, wherein the inner shield (80) includes an opening (84) that is configured to engage the flange locking element (18) and lock the inner shield (80) to the compartment (14).

12. The pen needle magazine (10) of claim 4, further comprising:
a compartment door (20) including a snap locking member (122); wherein
one of the plurality of compartments (14) includes a stepped locking element (116) that is a flexible member; and
in a closed, locked position, the stepped locking element (116) is rigid to engage the snap locking member (122) and lock the compartment door (20) to the compartment (14).

13. The pen needle magazine (10) of claim 12, wherein
in a closed, unlocked position, the stepped locking member (116) elastically deforms to disengage the snap locking member (122) and unlock the compartment door (20) from the compartment (14).

14. The pen needle magazine (10) of claim 4, further comprising:
a seal (30) disposed on a top surface of each of the plurality of compartments (14) to enclose each of the pen needles (40) in a corresponding compartment (14); and
a plurality of compartment doors (20) that each cover the corresponding compartment (14).

15. The pen needle magazine (10) of claim 14, wherein each seal (30) is disposed between the top surface of one of the plurality of compartments (14) and a corresponding compartment door (20) in a closed, unlocked position.

16. The pen needle magazine (10) of claim 4, further comprising:
a seal (30) including a sealing portion (32) and a tab portion (34);
the plurality of compartments (14) each having a stepped locking element (16); and
a plurality of compartment doors (20) each having a snap locking member (22) that is configured to engage the stepped locking element (16); wherein
in a closed, unlocked position of one of the plurality of compartments (14), the sealing portion (32) is disposed between a top surface of one of the plurality of compartments (14) and the corresponding compartment door (20), and the tab portion (34) is disposed between the stepped locking element (16) and the snap locking member (22).

17. The pen needle magazine (10) of claim 16, wherein in a closed, locked position of one of the plurality of compartments (14), the seal (30) is removed and the stepped locking element (16) engages the snap locking member (22).

18. The pen needle magazine (10) of claim 16, wherein in an open, unlocked position of one of the plurality of compartments (14), the seal (30) is removed from the top surface of the compartment (14) to access the pen needle (40) and simultaneously open the compartment (14).

19. The pen needle magazine (10) of claim 16, further comprising a tamper resistant member (19) that shields the seal (30), the stepped locking element (16) and the snap locking member (22) from accidental operation.

20. A pen needle magazine (10) comprising:
a plurality of compartments (14) each including an inner thread (82), each compartment (14) carrying a pen needle (40) with an inner thread (44) and an outer thread (46); wherein
the inner thread (82) is configured to engage the outer thread (46) of the pen needle (40); and
the inner thread (44) and the outer thread (46) of the pen needle (40) are threaded in different directions.

21. A method of using a plurality of pen needles (40) stored in a housing (12), the method comprising:
connecting an unused pen needle (40) to a medication delivery pen (2) for medication delivery;
removing the unused pen needle (40) from a cavity of the housing (12) simultaneously with the connecting step;
administering medication via the pen needle (40) connected to the medication delivery pen (2);
disposing of the used pen needle (40) after medication delivery into the cavity of the housing (12) and simultaneously disconnecting the used pen needle (40) from the medication delivery pen (2); and
sending the housing (12) to a manufacturer or a waste management entity after use of all of the plurality of pen needles (40) in the housing (12).

22. The method of claim 21, wherein a component of the housing (12) is sterilized and reused.
